(19) United States
(12) Patent Application Publication
(10) Pub. No.: US 2019/0046550 A1
(43) Pub. Date: Feb. 14, 2019

(54) TOPICAL COMPOSITION FOR TREATMENT AND/OR PROPHILAXIS OF MASTITIS AND/OR EDEMA

(71) Applicants: Dragan GAVRANOVIC, Samobor (HR); Dragana JOVIC, Dec-Srem (RS)

(72) Inventors: Dragan GAVRANOVIC, Samobor (HR); Dragana JOVIC, Dec-Srem (RS)

(21) Appl. No.: 16/079,602

(22) PCT Filed: Feb. 3, 2017
(86) PCT No.: PCT/HR2017/000001

§ 371 Date: Aug. 24, 2018

(30) Foreign Application Priority Data
Feb. 24, 2016 (HR) P20160195A

(51) Int. Cl. A61K 31/7016; A61K 9/00; A61P 31/04; A61K 31/19; A61K 9/08; A61K 47/44; A61K 35/57

(52) U.S. Cl. CPC A61K 31/7016; A61K 9/0041; A61P 31/04; A61K 35/57; A61K 9/08; A61K 47/44; A61K 31/19

(57) ABSTRACT

The present invention relates to a topical composition which is mixture of natural ingredients of plant and animal origin, to the process of its preparation, to its use for treatment and/or prophylaxis of mastitis and/or edema in female mammals, more particularly to its use for treatment and/or prophylaxis of mastitis and/or udder edema in dairy animals. The topical composition according to the invention is mixture of natural ingredients of plant and animal origin, wherein the topical composition comprises: sugar, carboxylic acid solution, egg white and vehicle.

### FIELD OF THE INVENTION

The present invention relates to a topical composition which is mixture of natural ingredients of plant and animal origin, to the process of its preparation, to its use for treatment and/or prophylaxis of mastitis and/or edema in female mammals, more particularly to its use for treatment and/or prophylaxis of mastitis and/or udder edema in dairy animals.

### BACKGROUND OF THE INVENTION

Care and maintenance of the teats and udders of dairy animals is essential in order to prevent damage to the animals and economic loss to the dairy farmer. Various inflammations of mammary glands, udder tissue and teats, particularly mammary glands, teat canal and milk ducts (galactophore) infections, in dairy animals, such as mastitis and/or udder edema, can result in increased veterinary costs, loss of milk production, and, in serious cases, death of the dairy animal.

Mastitis is the persistent, inflammatory reaction of the mammary gland and udder tissue, and is a major endemic disease of dairy cattle. It usually occurs when white blood cells (leukocytes) are released into the mammary gland, as an immune response to bacterial invasion of the teat canal by variety of bacterial sources, as well as by yeasts and moulds present on the farm. In some cases, the causes of mastitis are unknown and could occur as a result of chemical, mechanical, or thermal injury (such as weather extremes). Milk-secreting tissue and various ducts throughout the mammary glands are damaged due to toxins released by the bacteria. Although mastitis, in particular dairy animal mastitis, can be caused by many different bacterial species, the most common are the Staphylococcus, Streptococcus species and Escherichia coli. There are number of factors that may influence the emergence of this disease among which are: animal farming system, diet, in particular hygiene of premises where the animals are bred, hygiene of milking apparatus and axillary milking equipment, etc.

Mastitis may be classified according to the clinical symptoms to: acute clinical, hyper-acute clinical, sub-acute clinical, sub-clinical, chronical, gangrenous, and according to the mode of transmission to: contagious and environmental mastitis. Clinical mastitis has clear signs, such as edema, increase in temperature, hardening, pain in the mammary gland(s), grains, or any change in milk characteristics. In the subclinical form of mastitis, no macroscopic changes are seen, but rather changes in milk composition, i.e. there are no visible signs of udder inflammation.

Mastitis is a global problem for female mammals, in particular for dairy animals, such cows, sheep, goats and a like. Mastitis is considered as the disease causing the largest economic loss to milk production, due to the reduction of quantity and compromise to the quality of the produced milk, or even for the full loss of secreting capacity of the mammary gland(s). The treatment is possible with long-acting antibiotics, but milk from such dairy animals is not marketable for human consumption until drug residues have left the animal's system.

Udder edema, or caked bag, is the swelling of the udder and surrounding structures due to retention of fluid just prior to and during calving. Some edema is natural to the process of pregnancy and birthing, but excessive swelling can impair the functioning of the udder and ultimately damage it. The mechanism for udder edema is not precisely known but is thought to be a combination of heredity, nutrition prior to calving and metabolic changes natural to pregnancy. Udder edema is frequently seen in heifers, cows calving for the first time. This could be due to immaturity in the vascular structure of the udder causing it to be more vulnerable to fluid retention. Treatment includes massages and hot compresses on the affected areas. This stimulates blood flow which aids in the removal of the excess fluid. According to Merck's Veterinary Manual diuretics and corticosteroids have also proved useful in reducing the edema.

One approach to caring for the udder and teats in dairy animals is the topical application of ointment compositions in order to maintain skin quality and reduce infections. Usually such ointment compositions contain drugs, i.e. antibiotics or corticosteroids as active principles causing long lasting delay in milking until drug residues are not totally removed, which is particularly unacceptable for organic farming.

Consequently, there is an ongoing and unmet need for new, effective, low cost and environmentally acceptable methods and compositions useful for treatment and/or prophylaxis of mastitis and/or edema in female mammals, more particularly useful for treatment and/or prophylaxis of mastitis and/or udder edema in dairy animals, such as cows, sheep, goats and a like. In particular, there is a need for a low-cost and environmentally acceptable composition useful for treatment and/or prophylaxis of mastitis and/or edema in female mammals, more particularly useful for treatment and/or prophylaxis of mastitis and/or udder edema in dairy animals which is entirely based on natural ingredients and therefore acceptable in organic farming. Not less important, application of the non-antibiotic composition which is entirely based on natural ingredients is particularly desirable in order to antibiotic resistance development. The present invention addresses these needs.

### SUMMARY OF THE INVENTION

The present invention relates to a topical composition which is mixture of natural ingredients of plant and animal origin, to the process of its preparation, to its use for treatment and/or prophylaxis of mastitis and/or edema in female mammals, more particularly to its use for treatment and/or prophylaxis of mastitis and/or udder edema in dairy animals, such as cows, sheep, goats and a like.

In one aspect, the present invention relates to a topical composition which is mixture of natural ingredients of plant and animal origin, wherein the topical composition comprises: sugar, carboxylic acid solution, egg white and vehicle.

In a further aspect, the invention relates to the process for preparation of the topical composition according to the present invention, wherein process comprises steps:

a) adding powdered sugar portion-wise to vehicle under vigorous stirring to obtain homogenous mixture,
b) adding egg whites foam in small portions to the above prepared homogenous mixture with continued stirring,
c) adding carboxylic acid solution under intensive stirring to obtain uniform topical composition, and, if desired,
d) adding essential oil and/or further pharmaceutically acceptable excipients.

In another aspect, the invention relates to a topical composition which is mixture of natural ingredients of plant and animal origin according to the present invention, for use in medicine, in particular for use in veterinary medicine.

In another aspect, the invention relates to a topical composition which is mixture of natural ingredients of plant and animal origin according to the present invention, for use in the treatment and/or prophylaxis of mastitis and/or edema in female mammals, more particularly to its use for treatment and/or prophylaxis of mastitis and/or udder edema in dairy animals.

In yet another aspect, the invention relates to the use of a topical composition which is mixture of natural ingredients of plant and animal origin according to the present invention for the manufacture of a medicament for the treatment and/or prophylaxis of mastitis and/or edema in female mammals, more particularly for the treatment and/or prophylaxis of mastitis and/or udder edema in dairy animals.

In even further aspect, the invention relates to the method of treatment and/or prophylaxis of mastitis and/or edema in female mammals, more particularly to the method of treatment and/or prophylaxis of mastitis and/or udder edema in dairy animals by administering of an effective amount of a topical composition according to the present invention.

In another aspect, this invention provides method of treatment and/or prophylaxis of a female mammal subject, in particular dairy animals, susceptible to or afflicted with mastitis and/or edema, more particularly with mastitis and/or udder edema, which method comprises administering a therapeutically effective amount of a topical composition according to the present invention to a female mammal subject in need thereof.

Other objects and advantages will become apparent to those skilled in the art from a consideration of the ensuing detailed description.

### DETAILED DESCRIPTION OF THE INVENTION

### Definitions

It will be understood that the present invention covers all combinations of aspects, suitable, convenient and preferred groups described herein.

The term “sugar” refers to sweet, short-chain, soluble carbohydrate, which is composed of carbon, hydrogen, and oxygen, that may be derived from different sources and is disaccharide sugar including saccharose (table sugar), maltose and lactose or monosaccharide sugar including glucose (dextrose), fructose, galactose and mixtures thereof. Suitably the term refers to the saccharose.

The term “saccharose” or “table sugar” refers to naturally occurring carbohydrate sucrose which is a disaccharide combination of the monosaccharides glucose and fructose with the formula C₁₂H₂₂O₁₁, and found in many plants and plant parts. Suitably the term “saccharose” or “table sugar” refers to cane sugar (i.e. sucrose obtained from sugar cane either in raw or refined form) or beet sugar (i.e. sucrose obtained from sugar beet either raw or refined form), brown sugar (containing rests of syrup (molasses) which causes the colour), preserving sugar (coarse-grained, refined pure sugar), or icing sugar (very finely ground crystals with a touch of starch to prevent lumps), and a like. Suitably it is sucrose from sugar beet.

The term “carboxylic acid solution” refers to the 5% to 20% aqueous solution where acid is selected from acetic acid, citric acid, malic acid, fumaric acid, tartaric acid, lactic acid, gluconic acid, succinic acid, formic acid, propionic acid, butyric acid and mixtures thereof. Suitably the term refers to the acetic acid solution.

The term “acetic acid solution” or “Acidum Aceticum solution” refers to the solution of acetic acid (CH₃COOH) in water having from about 5% to about 20% of acetic acid, including vinegar and its variations obtained by transformation of wine or fruit juice to vinegar: such as white and red wine vinegar, apple cider vinegar, cane vinegar (sugar cane juice), coconut vinegar, rice vinegar, date vinegar, distilled (spirit vinegar, such as vinegar from malt or corn, or wood products vinegar from beech), Chinese black vinegar made from rice, wheat, millet, sorghum, or a combination thereof), vinegar made from honey, Job's tears vinegar, malt vinegar, pomegranate vinegar, vinegar made from raisins (called khall 'inab), and a like and mixtures thereof. Preferably the term refers to the natural vinegar solution used for human consumption (suitably white vinegar) having from about 5% to about 10% of acetic acid in water (preferably having 9% of acetic acid in water.

The term “egg white” or “ovum albo” refers to the egg white of poultry or fowl origin including egg white of hens (chickens), turkeys, domestic ducks, domestic geese, and a like and mixtures thereof. Suitably the term “egg white” refers to the hen or turkeys egg white, suitably it refers to the hen egg white. Said egg white may be freshly prepared or may be in a dried form as a powder.

The term “vehicle” refers to the pharmaceutically acceptable vehicle (excipient) of the natural origin used as a base for a topical composition such as ointment, unguent or balm: including pork fat (lard), animal wax, vegetable wax, paraffin wax, petroleum jelly (vaseline), vegetable oil or mixtures thereof. Suitably it refers to the pork fat, animal wax, petroleum yelly or mixtures thereof, preferably it refers to pork fat.

The term “adeps suillus” or “axungia porci” refers to the pork fat (lard), which may be industrially produced and may be hydrogenated to improve its stability at room temperature. It may be treated with bleaching and deodorizing agents, emulsifiers, and antioxidants such as BHT (butylated hydroxytoluene) to make it more consistent and prevent spoilage.

The term “animal wax” refers to the wax of animal origin including bee wax (yellow and white, suitably yellow), lanolin (wool wax), Chinese wax (produced by insect Ceroplastes ceriferus), Shellac wax (from the lac insect Kerria lacca) and mixtures thereof. Suitably it refers to the bee wax or lanolin.

The term “vegetable wax” refers to the wax of vegetable origin including candelilla wax, jojoba wax, carnauba wax, bayberry wax (from the surface wax of the fruits of the bayberry shrub, Myrica faya), Japan wax (a vegetable triglyceride not a true wax from the berries of Rhus and Toxicodendron species), rice bran wax (obtained from rice bran Oryza sativa), soy wax, Tallow Tree wax (from the seeds of the tallow tree Triadica sebifera), and mixtures thereof. Suitably it refers to the candelilla wax or carnauba wax.

The term “vegetable oil” or “vegetable fats” refers to a triglycerides extracted from a plant (fruits or seeds of plants) that are used for cosmetics and for medical purposes including olive oil, coconut oil, cocoa butter, almond oil, brazil nut oil, cashew oil, hazelnut oil, macadamia oil, mongongo nut oil, avocado oil, grapefruit seed oil, lemon oil, orange oil, apricot oil, amaranth oil, apple seed oil, black seed oil (from Nigella salvia), evening primrose oil, flaxseed oil, Dika oil (from Irvingia gabonensis seeds), papaya seed oil, nutmeg butter, perilla seed oil, Seje oil (from the seeds of Jessenia bataua), shea butter, tea seed oil (Camellia oil), wheat germ oil, and mixtures thereof. Suitably it refers to the olive oil, coconut oil or cocoa butter.

The term “essential oils” or “volatile oils” refers to the peppermint oil, Balsam of Peru (derived from the Myroxylon), eucalyptus oil, rose oil, and a like, and to mixtures thereof. The term “comprise”, and variations such as “comprises” and “comprising”, throughout the specification and the claims which follow, unless the context requires otherwise, will be understood to imply the inclusion of a stated integer or step or group of integers but not to the exclusion of any other integer or step or group of integers or steps.

The term “consists”, and variations such as “consisting”, throughout the specification and the claims which follow, unless the context requires otherwise, will be understood to imply the inclusion of a stated integer or step or group of integers but not to the inclusion of any other integer or step or group of integers or steps.

The term “mastitis” including puerperal and non-puerperal mastitis refers to the inflammatory reaction of breast or udder tissue which may be classified as milk stasis, non-infectious or infectious inflammation and abscess, resulting from inflammatory reaction of the mammary gland and/or udder tissue (including milk ducts such in case of galactophoritis), in particular it refers to mammary gland and/or udder infection when white blood cells (leukocytes) are released into the mammary gland, teat canal and udder tissue, usually in response to pathogens such as bacteria, yeast or mould invading the teat canal and/or udder tissue or as a result of chemical, mechanical, or thermal injury.

The term “udder edema” or “caked bag” refers to the swelling of the udder and surrounding structures due to retention of fluid just prior to and during calving.

The term “galactophoritis” refers to inflammation of the lobular and lactiferous duct system, mainly resulting from secretory disease and retention syndrome which may develop to the chronic inflammation of the milk ducts in the breast (galactophore).

The term “female subject” or “female mammal” to be treated may mean either a human or non-human animal.

The term “non-human animal” refers to the non-human milk-producing mammal, in particular dairy animal such as cows, sheep, goats, horse, buffalo, camel or yak, in particular the term refers to cows, sheep and goats, suitably to cows.

The term “effective amount” means the amount of a topical composition according to the present invention that, when administered to a subject for the treatment of mastitis and/or edema, more particularly for the treatment of mastitis and/or udder edema, is sufficient to affect such treatment. The “effective amount” can vary depending on the clinical symptoms and the age, weight, etc., of the subject.

The term “one or more” refers to one to four. In one embodiment, it refers to one to three.

In further embodiment, it refers to one or two. In a yet further embodiment, it refers to one.

The term “prophylaxis” is related to maintenance therapy as well as to prevention.

The term “maintenance therapy” refers to a preventive therapy that follows successful initial treatment of the acute phase of the illness where regular (usually smaller) doses of a topical composition according to the present invention are applied to the subject to prevent recurrence and worsening of the mammary gland and/or teat canal infections, more particularly to prevent recurrence and worsening of the mastitis and/or udder edema. Whereas “prevention” refers to a measure or procedure the purpose of which is to prevent, rather than to treat or cure mammary gland and/or teat canal infections, more particularly to prevent, rather than to treat or cure mastitis and/or udder edema.

“Preventing” or “prevention” refers to a reduction in risk of acquiring or developing the mastitis and/or edema, more particularly mastitis and/or udder edema (i.e., causing at least one of the clinical symptoms to occur in a subject that may be exposed to mammary gland and/or teat canal inflammation, more particularly to mastitis and/or udder edema-causing agent, or predisposed to the mammary gland and/or teat canal infections, more particularly to the mastitis and/or udder edema in advance of its onset).

“Treating” or “treatment” of mastitis and/or edema (of breast or udder in mammal), more particularly “treating” or “treatment” of mastitis and/or udder edema refers, in one embodiment, to ameliorating the disease and/or condition (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease and/or condition, either physically, (e.g., stabilization of a discernible symptom), physiologically, (e.g., stabilization of a physical parameter), or both. In a further embodiment, “treating” or “treatment” relates to slowing the progression of the disease and/or condition.

The “w %” abbreviation refers to a mass fraction of individual components in the composition mixture, expressed as a percentage of the mass of the individual component of the mixture in the total mass of the composition mixture.

### Embodiments of the Invention

The present invention is based on the identification that by using a topical composition which is mixture of natural ingredients of plant and animal origin, wherein the topical composition comprises: sugar, carboxylic acid solution, egg white and vehicle for treatment and/or prophylaxis of mastitis and/or edema in female mammals, more particularly of mastitis and/or udder edema in dairy animals, there are no undesired contraindications due to the fact all ingredients are of natural origin (i.e. without synthetic additives, preservatives, antibiotics and steroids or other chemicals), it is applicable for organic farming, and milk from such dairy animals can be used for human consumption immediately after somatic cell count reaches the allowed ranges (determined for example using California Mastitis Test (https://en.wikipedia.org/wiki/California_mastitis_test) and/or Porta SCC Test according to manufacturers instructions). In addition, the topical composition according to the present invention is readily prepared from easily available low-cost ingredients and can be simply applied.

Thus, the present invention relates to a topical composition, which is mixture of natural ingredients of plant and animal origin, to the process of its preparation, to its use for treatment and/or prophylaxis of mastitis and/or edema in female mammals, more particularly to its use for treatment and/or prophylaxis of mastitis and/or udder edema in dairy animals, such as cows, sheep, goats and a like (suitably cows).

In the first aspect, the present invention relates to a topical composition which is mixture of natural ingredients of plant and animal origin, wherein the topical composition comprises: sugar (preferably saccharose), carboxylic acid solution (preferably acetic acid solution), egg white and vehicle (suitably pork fat, animal wax or petroleum yelly; preferably pork fat). In another embodiment, the present invention relates to a topical composition which is mixture of natural ingredients of plant and animal origin, wherein the topical composition consist of: sugar (preferably saccharose), carboxylic acid solution (preferably acetic acid solution), egg white and vehicle (suitably pork fat, animal wax or petroleum yelly; preferably pork fat). In further embodiment, the present invention relates to a topical composition which is mixture of natural ingredients of plant and animal origin, wherein the topical composition consists of: 20-40 w % sugar (suitably saccharose), 5-20 w % carboxylic acid solution (preferably acetic acid solution), 5-20 w % egg white and 30-50 w % vehicle (suitably pork fat, animal wax or petroleum yelly; preferably pork fat). In yet further embodiment, the present invention relates to a topical composition which is mixture of natural ingredients of plant and animal origin, wherein the topical composition consists of: 25-30 w % sugar (preferably saccharose), 15-20 w % carboxylic acid solution (preferably acetic acid solution), 10-15 w % egg white and 35-40 w % vehicle (suitably pork fat, animal wax or petroleum yelly; preferably pork fat). Suitably the topical composition according to the present invention is consisting of 25-30 w % saccharose, 15-20 w % acetic acid solution having from about 5% to about 10% of acetic acid in water, 10-15 w % egg white and 35-40 w % pork fat.

In one aspect, the topical composition according to the present invention further comprises one or more essential oils or mixtures thereof in 0.1-1 w %. In one embodiment, the topical composition according to the present invention further comprises one or more essential oils or mixtures thereof in 0.4-1 w %. Suitably essential oil is peppermint oil or eucalyptus oil (preferably it is peppermint oil).

In a further aspect, the invention relates to the process for preparation of the topical composition according to the present invention, wherein process comprises steps:

a) adding powdered sugar portion-wise to vehicle under vigorous stirring to obtain homogenous mixture,
b) adding egg whites foam in small portions to the above prepared homogenous mixture with continued stirring,
c) adding carboxylic acid solution under intensive stirring to obtain uniform topical composition, and, if desired,
d) adding essential oil and/or further pharmaceutically acceptable excipients.

In another aspect, the invention relates to a topical composition which is mixture of natural ingredients of plant and animal origin according to the present invention, for use in medicine, in particular for use in veterinary medicine.

In a further aspect, the invention relates to a topical composition which is mixture of natural ingredients of plant and animal origin according to the present invention, for use in the treatment and/or prophylaxis of mastitis and/or edema (of breast or udder) in female mammals, more particularly to its use for treatment and/or prophylaxis of mastitis and/or udder edema in dairy animals. In another embodiment, the invention relates to a topical composition which is mixture of natural ingredients of plant and animal origin according to the present invention, for use in the treatment of mammary gland and/or udder tissue (including teat canal, milk ducts, skin and teat orifices) inflammations, more particularly to its use for treatment of mastitis and/or udder edema in dairy animals.

In yet another aspect, the invention relates to the use of a topical composition which is mixture of natural ingredients of plant and animal origin according to the present invention for the manufacture of a medicament for the treatment and/or prophylaxis of mastitis and/or edema (of breast or udder) in female mammals, more particularly for the treatment and/or prophylaxis of mastitis and/or udder edema in dairy animals. In another embodiment, the invention relates to the use of a topical composition which is mixture of natural ingredients of plant and animal origin according to the present invention for the manufacture of a medicament for the treatment of mammary gland and/or udder tissue (including teat canal, milk ducts, skin and teat orifices) inflammations, more particularly for the treatment of mastitis and/or udder edema in dairy animals.

In even another aspect, the invention relates to the methods of treatment and/or prophylaxis of mastitis and/or edema (of breast or udder) in female mammals, more particularly to the methods of treatment and/or prophylaxis of mastitis and/or udder edema in dairy animals by administering of an effective amount of a topical composition according to the present invention. In another embodiment, the invention relates to the methods of treatment of mammary gland and/or udder tissue (including teat canal, milk ducts, skin and teat orifices) inflammations, more particularly to the methods of treatment of mastitis and/or udder edema in dairy animals by administering of an effective amount of a topical composition according to the present invention.

In yet another aspect, this invention provides methods of treatment and/or prophylaxis of a female mammal subject, in particular dairy animals, susceptible to or afflicted with mastitis and/or edema (of bras or udder), more particularly with mastitis and/or udder edema, which methods comprise administering a therapeutically effective amount of a topical composition according to the present invention to a female mammal subject in need thereof. In another embodiment, this invention provides methods of treatment of dairy animals, susceptible to or afflicted with mammary gland and/or udder tissue (including teat canal, milk ducts, skin and teat orifices) inflammations, more particularly with mastitis and/or udder edema, which methods comprise administering a therapeutically effective amount of a topical composition according to the present invention to diary animal subject in need thereof.

In another aspect, the topical composition of the invention can be used as a support-combination therapy in animals with mammary gland infection which are under the intra-mammary and/or systemic antibacterial therapy in order to accelerate the healing process of the mammary glands.

In one aspect, female mammal to be treated according to the invention is human.

In another aspect, female mammal to be treated according to the invention is non-human animal. In further embodiment, non-human animal is dairy animal selected from cows, sheep, goats, horse, buffalo, camel and yak, in particular it is cow, sheep and goat, more particularly it is cow (including heifer).

The topical composition according to the present invention can be formulated as an ointment, unguent, balm, paste or cream, and topically applied in any manner. For instance, the composition can be applied by lubricating and massaging the teat.

In preferred embodiments, the topical composition according to the present invention provide antibacterial effect when applied to the breast or udder skin of a female mammal, especially the skin of the udder and teat of a dairy animal. As mentioned, it is believed that this effect is primarily the result of the combined action of the sugar, carboxylic acid solution and egg white, combined with vehicle (base for a topical composition) of natural origin (such as pork fat, animal wax or petroleum yelly; preferably pork fat) which in addition has moisturizing effect to the inflamed skin.

The topical composition according to the invention may be applied locally as a topical preparation by external lubrication of the skin in a thin layer for treatment and/or prophylaxis of mammary gland and/or udder tissue inflammations (including teat canal, milk ducts, skin and teat orifices inflammations), more particularly for treatment and/or prophylaxis mastitis and/or udder edema, specifically for treatment and/or prophylaxis of mastitis and/or udder edema.

Operating instructions: Topical composition is for local application: to be lubricated in a thin layer to the clean and dry skin, if desired skin may be previously disinfected. The use should be started as soon as the first signs of inflammation of the mammary gland and/or udder tissue (including teat canal, milk ducts, skin and teat orifices inflammations), more particularly for treatment and/or prophylaxis mastitis and/or udder edema, specifically for treatment and/or prophylaxis of mastitis and/or udder.

The composition described herein can be provided in any suitable container. In one embodiment, the container is plastic or glass container suitable for storing the composition.

The illustration of the present invention, is provided by following, non-limiting examples.

### EXAMPLES

### Example 1

### Preparation of Topical Composition

All used ingredients are natural ingredients of plant and animal origin and are commercially available.

### Composition 1:

To the enamelled container saccharose (300 g) was added and pulverized to the dispersibility degree that provides good homogeneity. In another container pork fat (400 g) was measured and then sequentially added to the container with saccharose in small aliquots, each aliquot was vigorously rubbed into the saccharose with continuous agitation. Then, foam of whipped egg whites (150 g, three chicken egg whites) was added in small portions to the above prepared homogenous mixture. Finally, to the obtained mixture 9% acetic acid solution (150 g) was added under intensive stirring to achieve uniform topical composition 1.

The resulting topical composition was transferred by spatula to a plastic container (500 g-1 kg) to remain protected from environmental factors which may cause spoiling. It is recommendable to store the composition in a dry, cool place protected from light.

### Composition 2:

Method A: To the pork fat (400 g) previously powdered saccharose (300 g) was added in portions under vigorous stirring. Then, foam of whipped egg whites (150 g, three chicken egg whites) was added in small portions to the above prepared homogenous mixture under vigorous stirring. Finally, to the obtained mixture 9% acetic acid solution (200 mL, 202 g) was added under intensive stirring to achieve uniform topical composition 2.

Method B: The same amounts of pork fat, saccharose and 9% acetic acid solution were used as in Composition 2, Method A but instead of fresh egg whites egg white powder was used (10.5 g). Firstly, homogenous mixture of saccharose with pork fat was prepared. Then egg white foam was prepared by adding aliquot of 9% acetic acid solution (50 to 70 mL) to egg white powder under vigorous stirring. Thus, prepared egg white foam was added to the homogenous mixture of saccharose and pork fat under the intensive stirring. Finally, the remaining aliquot of 9% acetic acid solution (i.e. up to 200 mL) was added under intensive stirring to achieve uniform topical composition 2.

### Composition 3:

Method A: To the pork fat (400 g) previously powdered saccharose (300 g) was added in portions under vigorous stirring. Then, foam of whipped egg whites (150 g, three chicken egg whites) was added in small portions to the above prepared homogenous mixture under vigorous stirring. To the obtained mixture firstly 9% acetic acid solution (200 mL, 202 g) was added and then peppermint oil (5 g) under intensive stirring to achieve uniform topical composition 3.

Method B: The same amounts of pork fat, saccharose and 9% acetic acid solution were used as in Composition 3, Method A but instead of fresh egg whites egg white powder was used (10.5 g). Firstly, homogenous mixture of saccharose with pork fat was prepared. Then egg white foam was prepared by adding aliquot of 9% acetic acid solution (50 to 70 mL) to egg white powder under vigorous stirring. Thus, prepared egg white foam was added to the homogenous mixture of saccharose and pork fat under the intensive stirring. Finally, the remaining aliquot of 9% acetic acid solution (i.e. up to 200 mL) was added and then peppermint oil (5 g) under intensive stirring to achieve uniform topical composition 3.

Comparison composition X:

Previously powdered saccharose (300 g) was added in portions to pork fat (400 g) under the vigorous stirring. Then, foam of whipped egg whites (150 g, three chicken egg whites) was added in small portions to the above prepared homogenous mixture under the intensive stirring to achieve uniform topical comparison composition X.

### Example 2

### Application of the Topical Composition

Topical compositions 1, 2 and 3, as well as comparison composition X were applied in treatment of cows and heifers with mastitis and udder edema with goal of determining the effectiveness of each.

Topical compositions were applied twice daily after morning and evening milking. The whole udder including teats was lubricated (in average 100 g of ointment per individual treatment). Animals included in the study were not treated with antibiotics or other drug therapy.

California Mastitis Test (CMT test; information available at internet link https://en.wikipedia.org/wiki/California_mastitis_test) was used for detection of subclinical mastitis, whereas Porta SCC Test (according to manufacturer's instructions) was used for determining somatic cell count (SCC). Thus, animals with increased number of somatic cells were first detected by CMT and then milk samples from quarters positive at CMT test were subjected to Porta SCC Test.

### Mastitis Treatment Results:

30 Animals were treated with comparison composition X, composition 2 or composition 3: out of which 10 animals having clinical mastitis and 20 animals with subclinical mastitis.

Treatment with comparison topical composition X lasted 5 days.

Animals having mastitis which were treated with comparison composition X showed no improvement after 5 days' treatment, but even worsening of symptoms. Thus, after 5 days treatment with comparison composition X all mastitis symptoms were still present: skin was swollen and red, and number of somatic cells increased.

Therefore, after 5 days therapy with comparison composition X was stopped, and then treatment continued either using composition 2 or using composition 3 for 10 to 12 days in average, i.e. up to complete relief of visible symptoms combined with SCC decrease to allowable level.

Treatment with topical compositions 2 or 3 lasted in average 7 to 10 days, or when compositions 2 or 3 were applied subsequent to comparison composition X (due to the reason there was no improvement after 5 days' treatment with comparison composition X) treatment lasted in average 10 to 12, i.e. up to improvement or complete relief of visible symptoms.

Within first 2 days of treatment excretion of pathological secret combined with reduction in soreness and redness was observed. After 3 to 5 days clinical symptoms disappeared.

Continued treatment resulted in decreasing SCC number up to allowable level.

In several animals treatment with compositions 2 or 3 continued even after complete relief for preventive purposes.

Improvement in skin epithelization was observed during treatment with compositions 1, 2 and 3, as well as beneficial effect in wound and abscess treatment of udder skin.

Udder Edema Treatment Results: 10 Animals having udder edema were treated with compositions 1, 2 or 3. Topical compositions were applied 2-3 days before and 2-3 days after calving giving good results.

It was observed that continuous application of topical composition in pregnant animals which suffered from udder edema led to a decrease in swelling and pain. Also, in treated animals which suffered of udder edema after calving: the first milking was much easier, i.e. without rejection of milking unit, and higher amount of milk was obtained than in untreated animals.

In several animals topical compositions 1, 2 or 3 were applied as preventive therapy in peripartum period (3 days before and 3 days after calving) which showed excellent effect especially with first calving animals because udder edema did not appear at all.

In animals with inflammation of the mammary glands and changed milk for which the first symptoms were observed during pre-milking test (presence of clots) the application of the topical composition after two consecutive milking resulted in improvement of udder health and milk quality.

In severe cases, due to treatment with topical composition and udder massage it was possible to increase milking frequency and number of milking.

In addition, in animals with hyperkeratosis of the teat skin, especially teat orifices application of the topical composition 1, 2 or 3 showed to be effective: skin and teat tops become much softer (i.e. the degree of keratinization was reduced).

Effectives of the topical composition was also observed in animals which have dry and rough papilla skin and which showed to feel discomfort during mechanical milking. After treatment with the topical composition it was observed that milking lasted shorter than before treatment.

1-10. (canceled)

11. A topical composition which is mixture of natural ingredients of plant and animal origin, wherein the topical composition comprises: sugar, carboxylic acid solution, egg white and vehicle and wherein the topical composition is configured for use in the treatment and/or prophylaxis of mastitis and/or edema of breast or udder.

12. The topical composition according to claim 11, wherein mastitis and breast or udder edema comprises mammary gland and/or udder tissue inflammations including teat canal, milk ducts, skin and teat orifices inflammations,

13. A method of treatment and/or prophylaxis of mastitis and/or breast or udder edema by administering of an effective amount of a topical composition which is mixture of natural ingredients of plant and animal origin, wherein the topical composition includes: sugar, carboxylic acid solution, egg white and vehicle and wherein the topical composition is configured.

14. A method of treatment and/or prophylaxis of a female mammal subject susceptible to or afflicted with mastitis and/or breast or udder edema, comprising administering a therapeutically effective amount of a topical composition to a female mammal subject in need thereof wherein said topical composition is mixture of natural ingredients of plant and animal origin, wherein the topical composition includes: sugar, carboxylic acid solution, egg white and vehicle and wherein the topical composition is configured.

15. The topical composition according to claim 11 , wherein the topical composition consist of: sugar, carboxylic acid solution egg white and vehicle.

16. The topical composition according to claims 11, wherein the topical composition comprises: 20-40 w % sugar, 5-20 w % carboxylic acid solution, 5-20 w % egg white and 30-50 w % vehicle.

17. The topical composition according to claim 16, wherein the topical composition comprises: 25-30 w % sugar, 15-20 w % carboxylic acid solution, 10-15 w % egg white and 35-40 w % vehicle.

18. The topical composition of claims 11, wherein sugar is saccharose.

19. The topical composition of claims 11, wherein carboxylic acid solution is acetic acid solution.

20. The topical composition of claims 19, wherein the acetic acid solution having 9% acetic acid in water.

21. The topical composition of claims 11, wherein vehicle is pork fat, animal wax or petroleum jelly or mixtures thereof.

22. The topical composition of claims 11, wherein sugar is saccharose, carboxylic acid solution is 9% acetic acid solution, and vehicle is pork fat.
